(19) United States
(12) Patent Application Publication
(10) Pub. No.: US 2006/0141467 A1
(43) Pub. Date: Jun. 29, 2006

(54) Methods and kits for making double stranded ribonucleic acids

(75) Inventor: Brian Neil Zeiler, Washington Township, NJ (US)

Correspondence Address: DAVIDSON, DAVIDSON & KAPPEL, LLC, 485 SEVENTH AVENUE, 14TH FLOOR, NEW YORK, NY, 10018, US

(73) Assignee: Q-RNA, Inc., New York, NY (US)

(21) Appl. No.: 10/539,850

(22) PCT Filed: Dec. 18, 2003
(86) PCT No.: PCT/US03/40378

§ 371 Date: Nov. 18, 2005

Related U.S. Application Data
(60) Provisional application No. 60/435,919, filed on Dec. 20, 2002.

(51) Int. Cl. C12Q 1/68; C12P 19/34

(52) U.S. Cl. 435/6; 435/91.2

(57) ABSTRACT

Embodiments of the present invention are directed to methods and kits for the generation of double stranded RNA by RNA dependent RNA polymerases. The methods and kits feature exponential generation of such RNAs through simple steps/ The RNA is suitable for interference with expression in cells.

This application is a 35 U.S.C. 371 filing of International Application No. PCT/US03/040378, filed Dec. 18, 2003, which claims the benefit under 35 U.S.C. 119(e) of U.S. Provisional Application No. 60/435,919, filed Dec. 20, 2002.

### FIELD OF THE INVENTION

The present invention relates to the methods and kits for making double stranded ribonucleic acids. Double stranded ribonucleic acids have applications for blocking or controlling expression of selected genes. This technique for blocking or controlling expression of selected genes using double stranded ribonucleic acid is referred to as ribonucleic acid interference.

### BACKGROUND OF THE INVENTION

As used in the present application the term “RNA” refers to ribonucleic acid and “DNA” refers to a deoxyribonucleic acid. The genetic code is carried in the sequence of nucleotides comprising the DNA of living cells. Living cells make proteins by making an RNA copy, or transcript, of portions of such DNA encoding a gene. This RNA, messenger RNA, or simply mRNA, cases the message of the nature of the protein that will be made to the cell organelles engaged in protein synthesis. The process of making proteins, moving from the genetic code to the final protein, is referred to as “expression”.

Double stranded nucleic acid refers to nucleic acid that is paired with its complement through Watson Crick binding. The letters “ds” will be used to denote double stranded nucleic acid. Double stranded ribonucleic acids, dsRNAs, are used for RNA interference, or simply, RNAi. RNAi is a process for interfering with the expression of proteins by the cells engaged in protein synthesis. RNAi has applications for controlling expression in living cells, for use in cell culture and fermentation processes, and in therapies.

RNA interference (RNAi) is one of the oldest and most ubiquitous eukaryotic regulatory mechanisms known and only recently has its application as a research tool become fully realized (Maine, E. M., 2001; Ullu E et al, 2002; Hutvager and Zamore, 2002; Brantl 2002; Lindenbach and Rice, 2002). RNAi is a naturally occurring process in which the degradation of gene-specific cellular RNAs results from the introduction of homologous double-stranded RNAs or “silencer” RNAs (siRNAs). In this way, the expression of specific genes of interest can be precisely turned off by introducing siRNAs containing sequences derived from the target cellular RNA. This approach, called reverse genetic analysis, makes it possible to discover the function of previously unknown genes that may play a role in human health. Due to its specificity in gene targeting and compatibility with well-defined cell culture systems, RNAi is the method of choice for studying the vast number of available new gene sequences resulting from current genome sequencing projects (Ueda R, 2001). RNAi avoids the need for the costly and time-consuming process of generating knockout animals, thereby lowering the cost of genetic studies and making it possible to study organisms previously considered not to be open to genetic analysis.

Present methods of making such nucleic acids are time consuming, awkward, and expensive. Most methods involve generating RNA transcripts from DNA every time material is needed. This requires two separate rounds of synthesis, and a separate hybridization step. The method requires a significant amount of DNA template that itself must be made at regular intervals.

The term “amplify” is used herein in the sense of making more than one copy. Enzymatic chain reactions, of which the polymerase chain reaction (PCR) is one example, make multiple copies of a nucleic acid having a desired sequence. RNA dependent RNA polymerases use RNA as a template to generate copies of the template. This document will use the designation “RDRP” for RNA dependent RNA polymerase.

It would be advantageous to generate double-stranded RNAs (dsRNA) for use as siRNAs in RNAi experiments in a single reaction, with a single enzyme without further treatment. It would also be advantageous if RNA could be used as the template for this reaction, thereby alleviating the need for repeated preparation of DNA templates. These and other benefits and advantages are obtained with embodiments of the present invention which are summarized in the following section.

### SUMMARY OF THE INVENTION

Embodiments of the present invention are directed to methods and kits for generating double stranded RNA. One embodiment of the present invention is directed to a method of making double stranded RNA having a selected sequence comprising the step of forming an admixture of an RNA dependent RNA polymerase, reagents for the synthesis of transcript nucleic acids, and at least one template nucleic acid. The template nucleic acid acts as a template for the synthesis of RNA encoding the selected sequence upon the imposition of nucleic acid synthesis conditions and in the presence of said reagents and RNA dependent RNA polymerase. The method further comprises the step of imposing nucleic acid synthesis conditions on the admixture to form an amplification product comprising double stranded RNA encoding the selected sequence.

Preferably, the template nucleic acid is a deoxyribonucleic acid. And, preferably, RNA dependent RNA polymerase is Q-Beta replicase and modifications thereto. Other RNA dependent RNA polymerases comprise polymerases associated with turnip yellow mosaic virus, turnip crinkle virus, tobacco vein mottling virus, and hepatitus C virus, and NS5B protein and poliovirus30 pol protein.

The reaction product comprising double stranded RNA has applications for RNAi. That is, the dsRNA formed as an amplification product inhibits the expression of a selected gene in a cell.

Preferably, the template nucleic acid has portions represented by the formula:
5′ A-B-C 3′.
At least one letter A and C represents a sequence recognized by the RNA dependent RNA polymerase and at least one of A and C represents the antisense of said sequence recognized by the RNA dependant RNA polymerase. The letter B represents a sequence corresponding to the selected sequence or the antisense of the selected sequence.

The sequence represented in the formula above can be readily synthesized. That is, the sequence represented by A and C are synthesized with the sequence represented B. This nucleic acid can be cloned into suitable plasmids and other vectors for ease of use. In the alternative the sequence represented by A and C are cloned to the sequence represented by B.

Preferably, the template is a deoxyribonucleic acid. And, the admixture further comprises a DNA-dependent RNA polymerase, such as T7 RNA polymerase, SP6, T3, and RNA polymerase 1. The DNA-dependent RNA polymerase is used to transcribe the DNA template to make at least one RNA recognized by said RNA dependent RNA polymerase. The RNA dependent RNA polymerase generates an amplification product.

Preferably, the reagents for the synthesis of nucleic acid comprise modified nucleotides. For example, without limitation, preferred modified nucleotides have modifications at the number two position, such as 2′-amino, 2′-fluoro, 2′-azido, 2′Omethyl, 2′ ara.

A further embodiment of the present invention is directed to a kit for making double stranded RNA. The kit comprises an RNA dependent RNA polymerase which synthesizes double stranded nucleic acid in the presence of reagents and conditions suitable for nucleic acid synthesis. The kit further comprises reagents for the synthesis of transcript nucleic acids; and means for making at least one template nucleic acid. The template nucleic acid acts as a template for the synthesis of RNA encoding the selected sequence upon the imposition of nucleic acid synthesis conditions and in the presence of the reagents and RNA dependent RNA polymerase. The kit further comprises instructions for imposing nucleic acid synthesis conditions on said admixture to form an amplification product comprising double stranded RNA encoding the selected sequence. The kit would have individual components packaged in a conventional manner with vials containing reagents, buffers and the like boxed with instructions.

The double stranded RNA made can preferably be used for RNAi purposes.

Preferably, the means for making at least one template nucleic acid is a deoxyribonucleic acid. This DNA encodes sequences corresponding to the selected sequence.

Preferably, the RNA dependent RNA polymerase is Q-Beta replicase and modifications thereto. Q-Beta replicase and some modifications of such enzyme are, under certain conditions, capable of using DNA as a template.

Preferably, the template nucleic acid has portions represented by the formula:
5′ A-B-C 3′
wherein at least one letter A and C represents a sequence recognized by the RNA dependent RNA polymerase and at least one of A and C represents the antisense of said sequence recognized by the RNA dependant RNA polymerase The letter B represents a sequence corresponding to the selected sequence or the antisense of the selected sequence.

The sequence represented by A and C can be synthesized with the sequence represented B. Or, in the alternative, the sequence represented by A and C are cloned to the sequence represented by B. Cloning and synthesis are facilitated where the template is a deoxyribonucleic acid. The sequence represented by A-B-C can be readily maintained in plasmids.

Preferably, where the template is DNA said admixture further comprises a T7 RNA polymerase. The T7 RNA polymerase transcribes the template to make at least one RNA recognized by said RNA dependent RNA polymerase which RNA dependent RNA polymerase generates an amplification product.

During the making of the amplification product, the RNA formed may incorporate modified nucleotides. The reagents for the synthesis of nucleic acid may comprise modified nucleotides for such purpose. Preferably, the modified nucleotides have modifications at the number two position. By way of example without limitation modified nucleotides comprise limited 2′-amino, 2′-fluoro, 2′-azido, 2′Omethyl, 2′ ara.

Embodiments of the present invention allow the making of large quantities of dsRNA. RNA dependent RNA polymerases generate amplification products exponentially. The process featured in the present methods and kits have fewer steps and take shorter times to generate an amount of dsRNA.

These and other advantages will be apparent to those skilled in the art upon viewing the drawings and reading the brief description of the drawing, and the detailed description of the drawings which follow.

### BRIEF DESCRIPTION OF DRAWINGS

FIG. 1 depicts a kit embodying features of the present invention;

FIG. 2 depicts a schematic flow diagram of the making of a template nucleic acid by cloning;

FIG. 3 depicts a schematic flow diagram of the making of a template nucleic acid by PCR;

FIG. 4 depicts a schematic flow diagram of the direct synthesis of a template nucleic acid from a DNA oligonucleotide;

FIG. 5 depicts a schematic flow diagram of the amplification of an RNA template in accordance with features of the present invention and use of such amplification product for RNAi; and

### DETAILED DESCRIPTION OF THE INVENTION

RNA interference (RNAi) is one of the oldest and most ubiquitous eukaryotic regulatory mechanisms known and only recently has its application as a research tool become fully realized (Maine, E. M., 2001; Ullu E et al, 2002; Hutvager and Zamore, 2002; Brantl 2002; Lindenbach and Rice, 2002). RNAi is a naturally occurring process in which the degradation of gene-specific cellular RNAs results from the introduction of homologous double-stranded RNAs or “silencer” RNAs (siRNA). In this way, the expression of specific genes of interest can be precisely turned off by introducing siRNAs containing sequences derived from the target cellular RNA. This approach, called reverse genetic analysis, makes it possible to discover the function of previously unknown genes that may play a role in human health. Due to its specificity in gene targeting and compatibility with well-defined cell culture systems, RNAi is the method of choice for studying the vast number of available new gene sequences resulting from current genome sequencing projects (Ueda R, 2001). RNAi avoids the need for the costly and time-consuming process of generating knockout animals, thereby lowering the cost of genetic studies and making it possible to study organisms previously considered not to be open to genetic analysis.

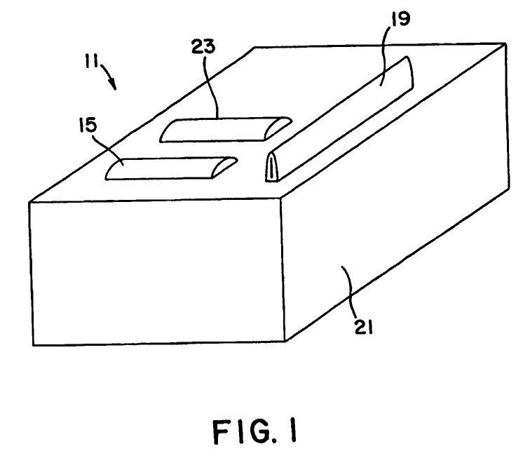

Embodiments of the present invention will be described with respect to a kit for making double stranded RNA. A kit embodying features of the present invention is depicted in FIG. 1, and is generally designated by the numeral 11. The kit comprises the following major elements: an RNA dependent RNA polymerase, contained in a suitable containment vessel 15, instructions 19 for the use of such polymerase for making RNAi compositions and packaging 21.

Containment vessels, such as containment vessel 15, may take different forms. Examples of containment vessels include vials, which are illustrated, envelopes, tins, ampules and the like. The contents of the containment vessels may be ready for use or dehydrated for reconstitution.

The enzyme is contained in vial 15, an RNA dependent RNA polymerase, synthesizes double stranded nucleic acid in the presence of reagents. Preferably, the kit 11 further comprises reagents in one or more additional containment vessels of which only one is illustrated, designated vial 23, for the synthesis of transcript nucleic acids; and means for making at least one template nucleic acid. The template nucleic acid acts as a template for the synthesis of RNA encoding the selected sequence upon the imposition of nucleic acid synthesis conditions and in the presence of the reagents and RNA dependent RNA polymerase.

The kit 11 further comprises instructions 19 for imposing nucleic acid synthesis conditions on an admixture of reagents to form an amplification product comprising double stranded RNA encoding the selected sequence. Preferably, the kit would instruct the user as to the use of the double stranded RNA for controlling expression in cellular processes.

The instructions would describe a method of making double stranded RNA having a selected sequence comprising the step o forming an admixture of an RNA dependent RNA polymerase, reagents for the synthesis of transcript nucleic acids, and at least one template nucleic acid. The template nucleic acid acts as a template for the synthesis of RNA encoding the selected sequence upon the imposition of nucleic acid synthesis conditions and in the presence of said reagents and RNA dependent RNA polymerase. The method further comprises the step of imposing nucleic acid synthesis conditions on the admixture to form an amplification product comprising double stranded RNA encoding the selected sequence.

Preferably, the template nucleic acid has portions represented by the formula:
5′ A-B-C 3′
wherein the at least one letter A and C represents a sequence recognized by said RNA dependent RNA polymerase and at least one of said A and C represents the antisense of said sequence recognized by said RNA dependant RNA polymerase. The letter B represents a sequence corresponding to the selected sequence or the antisense of the selected sequence.

The sequence represented by A and C can be synthesized with the sequence represented B. Or, in the alternative, the sequence represented by A and C are cloned to the sequence represented by B. Cloning and synthesis are facilitated where the template is a deoxyribonucleic acid. The sequence represented by A-B-C can be readily maintained in plasmids.

Preferably, where the template is DNA said admixture further comprises a T7 RNA polymerase. The T7 RNA polymerase transcribes the template to make at least one RNA recognized by said RNA dependent RNA polymerase which RNA dependent RNA polymerase generates an amplification product. The kit may further comprise the enzyme T7 RNA polymerase in the vial 15 or vial 23, or in a separate vial (not shown).

The kit 11 would have individual components packaged in a conventional manner, with vials 15 and 23, containing enzymes, reagents, buffers and the like boxed with instructions, in packaging 21. Packaging 21 is in the form of a box, but may take many forms including bags, wraps, bundles plastic formed containers and the like common in the industry.

The double stranded RNA made with kit 11 can preferably be used for RNAi purposes. Preferably, the instructions comprise directions for the RNAi purposes.

Embodiments of the present invention feature an enzyme, Q-Beta replicase, a powerful replicator of RNAs providing 10¹³ fold amplification within 30 minutes at room temperature. Q-beta replicase is a well-studied enzyme often studied for its robust RNA amplification properties (Biebricher C K, Eigen M, Gardiner W C Jr. Kinetics of RNA replication. Biochemistry. 1983 May 10; 22(10):2544-59). One of the products of Q-beta replication is double-stranded RNA (Biebricher, C K, Eigen, M, and Gardiner, W C. (1984) Kinetics of RNA replication: plus-minus asymmetry and double-strand formation. Biochemistry, 23, 3186-3194; Axelrod V D, Brown E, Priano C, Mills D R. Coliphage Q beta RNA replication: RNA catalytic for single-strand release. Virology; 1991 October; 184(2):595-608).

Protocals for the isolation and or manufacture of the enzyme are known in the art. A preferred enzyme is sold under the name of Q-Amp™ by Q-RNA, Inc. This enzyme has been modified to improve the manner in which it is isolated and purified. Preferably, the enzyme has no contamination from cellular RNAs endogenous to bacteria and has no detectable nuclease activities.

Q-Beta replicase amplification reactions are simple to set up, require a single tube and do not require special renaturation procedures. By way of example, without limitation, one set of conditions for forming amplification products with the enzyme Q-Beta replicase is 200 microM NTPs, 80 milliM TRIS-HCl, pH 7.5, 10 milliM MgCl₂, 2 milliM DTT. The reaction tolerates a wide range of salt conditions (eg. 0 to 500 milliM NaCl), can be initiated with small amounts of template (1 nanogram down to a single molecule) and Q-beta replicase may be present at a concentration of 50-200 nanoM.

Q-Beta replicase can incorporate modified nucleotides, enabling the synthesis of stabilized RNAs or RNAs with other functional groups. Under certain conditions Q-Beta replicase can use DNA as a template. Such use of DNA as a template can further simplify the generation of dsRNAs. Use of Q-Beta replicase is a significant advance over current techniques.

There are currently on the market kits for the express purpose of generating siRNA for RNAi experiments. Before using the kit, a researcher must first plan and design a few potential siRNA sequences and then order these sequences encoded in several synthetic oligonucleotides. The oligonucleotides must be assembled as templates for T7 transcription, followed by transcription reactions in separate tubes. The products of these reactions are then combined and annealed to form siRNAs. Although one synthesis should yield enough RNA for several reactions, eventually the procedure must be repeated in its entirety when the RNA stock runs out.

With Q-Beta replicase, a single transcription can generate an inexhaustible supply of RNA template. Only 1 pg or less is needed to initiate a reaction that can generate microgram amounts of dsRNA. Because both strands can be made in the same reaction vessel, there is no need for separate transcription reactions or additional annealing steps—the RNAs will anneal automatically as their concentrations increase. Therefore high yields of dsRNA can be generated in under thirty minutes.

It is also possible to combine T7 RNA polymerase and Q-Beta replicase in a single reaction. The simplest approach for generating siRNAs with RDRPs is to use DNA directly as a template. Although the efficiency of DNA as a template is about 1000-fold lower than RNA in some cases all that need be generated are enough RNA transcripts that can themselves be used as high efficiency templates. Again, all of these steps can occur in a single tube. Finally, an additional benefit of using RDRPs is that the products can also serve as templates. Therefore future rounds of in vitro transcription are no longer necessary—the reaction products from previous rounds of siRNA synthesis can be used as templates, thereby creating a virtually inexhaustible supply of template.

RNA can than be diluted and a single picogram would be used to seed the reaction. So 1 ug of synthesized RNA is enough for 10⁶ siRNA reactions with a RDRP. Because of the nature of RDRPs, there is never a need to repeat the initial RNA transcription because a RDRP can use its own amplification product as a template. Thus only a single synthesis of RNA template must be prepared for virtually a lifetime supply of template to generate dsRNA.

RDRP also have a lower level of activity on DNA templates. Our research shows that the sensitivity of amplification of DNA is roughly 1000-fold lower than with RNA as a template. Therefore if 1 picogram of RNA template is required to seed an amplification reaction, 1 ng of DNA will be needed on average. A typical synthesis of DNA oligos at the 250 nmole scale, the smallest scale commercially available, will yield hundreds of micrograms, which relates to thousands of amplification reactions. In essence, the DNA template becomes obsolete once enough RNA template is generated due to the higher efficiency of amplification. Therefore, one oligonucleotide synthesis per siRNA is all that is required in terms of construction of the siRNA template.

Use of the double stranded product as a siRNA will follow currently acceptable techniques. There are currently several kits on the market that can enable the transfection of siRNAs into cells. Our approach would take advantage of these pre-existing methods. There are two general approaches in the synthesis of siRNAs. One is to rationally target specific regions in a cellular RNA and to generate small RNAs to these regions. Typically several such constructs are made targeting different regions in a cellular RNA in hopes that at least one will show acceptable efficiency in terminating the expression of the target gene. Another approach is to generate a long dsRNA containing much of the cellular RNA. Such RNAs can either be introduced to cells, which process the dsRNA into ˜22 nucleotide siRNAs (Elbashir, S. M., Lendeckel, W., and Tuschl, T. 2000. RNA interference is mediated by 21- and 22-nucleotide RNAs. Genes and Development, 15:188-200). Another approach is to process the long dsRNA into smaller pieces before transfection into cells by digesting with a double stranded ribonuclease like Dicer (Grosshans, H and Slack, F J. 2002. The Journal of Cell Biology, 156(1): 17-21), E. coli RNase III, or other RNase III-like double-stranded ribonucleases (Lamontagne B et al. 2001. Curr Issues Mol. Biol. 3(4):71-8). dsRNAs generated by RDRPs are suitable for all of these techniques.

Further advantages of the present invention will be apparent from the description provided in the following Examples.

### EXAMPLE NUMBER 1

### Making a Template

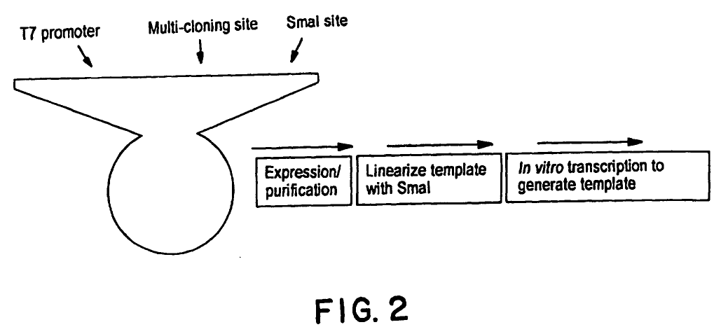

Example 1 describes the making of a template with reference to FIG. 2. FIG. 2 depicts a flow diagram representing steps in a process for making a template for use in making double stranded RNA.

Each step is performed in accordance with standard techniques known to individuals skilled in the art. FIG. 2 illustrates a plasmid with suitable restriction sites and a T7 promoter site. A nucleic acid having the formula:
5′ A-B-C 3′.
At least one letter A and C represents a sequence recognized by an RNA dependent RNA polymerase and at least one of said A and C represents the antisense of the sequence recognized by the RNA dependant RNA polymerase is cloned into the multi-cloning site. The letter B represents a sequence corresponding to the selected sequence or the antisense of the selected sequence.

Next, the plasmid in expressed in a suitable vector and purified. The plasmid in linearized with SmaI. The linearized nucleic acid is then transcribed to generate a template that is recognized by the RNA dependant RNA polymerase. As used herein the word “recognized” means that the enzyme uses such nucleic acid to generate further amplification product, either the plus or minus strand, of the template.

### EXAMPLE 2

### Making a Template with PCR Processes

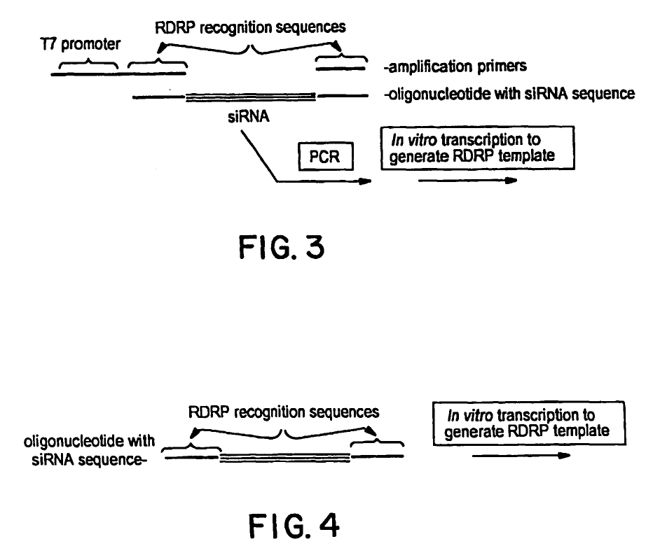

This Example 2 will describe the making of a template with PCR processes. For short templates, synthetic oligonucleotides may decrease the time of construction. With reference to FIG. 3, a DNA molecule is synthesized. A nucleic acid is synthesized having the formula, or its antisense:
5′ A-B-C 3′
At least one letter A and C represents a sequence recognized by an RNA dependent RNA polymerase and at least one of said A and C represents the antisense of the sequence recognized by the RNA dependant RNA polymerase is cloned. The letter B represents a sequence corresponding to the selected sequence or the antisense of the selected sequence. This sequence is denoted “siRNA” in the Figure.

The nucleic acid is amplified by PCR using primers which incorporate a T7 promoter sequence. The amplified DNA is transcribed with T7 polymerase to generate a transcription product. The transcription product is amplified with an RNA dependant RNA polymerase such as Q-Beta replicase.

### EXAMPLE 3

### Direct Synthesis of Template Nucleic Acid from DNA Oligonucleotide

This Example 3 describes the direct synthesis of double stranded RNA from a DNA oligonucleotide. Under certain conditions RNA dependant RNA polymerases may read DNA templates directly.

With reference to FIG. 4, a DNA molecule is synthesized having the formula:
5′ A-B-C 3′
At least one letter A and C represents a sequence recognized by an RNA dependent RNA polymerase and at least one of said A and C represents the antisense of the sequence recognized by the RNA dependant RNA polymerase is cloned. The letter B represents a sequence corresponding to the selected sequence or the antisense of the selected sequence. This sequence is denoted “siRNA” in the Figure.

The nucleic acid is amplified by Q-Beta replicase reading the DNA template directly. Amplification of DNA templates are similar to the amplification of RNA templates and by way of example, without limitation, one set of conditions for forming amplification products is 50-200 nanoM Q-Beta replicase, 200 microM NTPs, 80 milliM TRIS-HCl, pH 7.5, 10 milliM MgCl₂, 2 milliM DTT. The amount of DNA required to initiate amplification by Q-beta replicase is typically 100 to 1000-times more than its RNA counterpart. Therefore, at least 10⁷ strands of DNA should suffice for any template. After amplification, DNA templates can efficiently be removed from the reaction by deoxyribonucleases such as DnaseI.

### EXAMPLE 4

### Amplification of RNA by RNA Dependant RNA Polymerase

The RNA products of Examples 1-3 are amplifiable by RNA dependant RNA polymerases. One set of conditions for forming amplification products with the enzyme Q-Beta replicase is 100 nanoM Q-Beta replicase, 200 microM NTPs, 80 milliM TRIS-HCl, pH 7.5, 10 milliM MgCl₂, 2 milliM DTT. Conditions for amplification with other enzymes are as reported in the literature.

Due to manner in which RNA dependant RNA polymerases generate nucleic acid, the synthesis of template nucleic acid occurs exponentially. The enzymes recognize both the sense and antisense strands as templates. A single molecule can be amplified to a visible product in twenty minutes at room temperature.

### EXAMPLE 5

### Use of Double Stranded RNA Derived from Amplification for RNAi Purposes

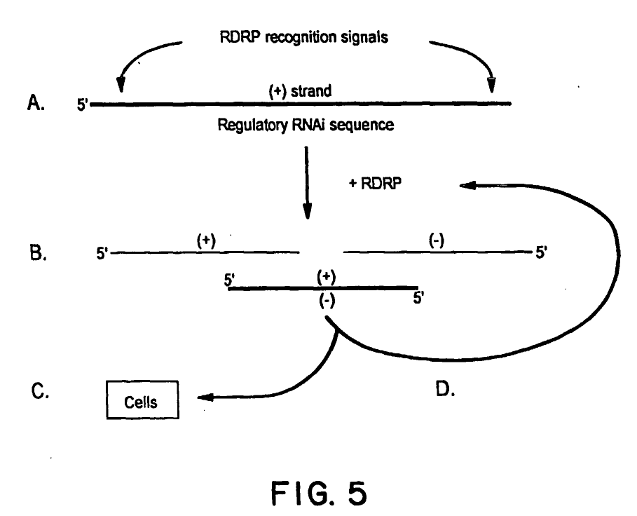

FIG. 5 depicts the amplification of RNA and its use in cells to control translation processes. RNA produced by amplification by the RNA dependant RNA polymerase is used for RNAi purposes as reported in the literature.

Thus, embodiments of the present invention have been described with respect to the examples, figures, and the detailed description contained herein with the understanding that individuals skilled in the art are able to make modifications and alterations to the present teaching. Therefore, the invention should not be limited to the specifics of such examples, figures and detailed description but should encompass the full scope of the subject matter of the following claims.

### REFERENCES

Axelrod V D, Brown E, Priano C, Mills D R. Coliphage Q beta RNA replication: RNA catalytic for single-strand release. Virology. 1991 October; 184(2):595-608.
Biebricher C K, Eigen M, Gardiner W C Jr. Kinetics of RNA replication. Biochemistry. 1983 May 10; 22(10):2544-59.
Brantl S. (2002) Antisense-RNA regulation and RNA interference. Biochim Biophys Acta. 3; 1575(1-3):15-25.
Elbashir, S. M., Lendeckel, W., and Tuschl, T. 2000. RNA interference is mediated by 21- and 22-nucleotide RNAs. Genes and Development, 15:188-200.
Hutvagner G, Zamore P D. (2002) A MicroRNA in a Multiple-Turnover RNAi Enzyme Complex. Science [epub ahead of print].
Lamontagne B, Larose S, Boulanger J, Elela S A. (2002) The RNase III family: a conserved structure and expanding functions in eukaryotic dsRNA metabolism. Curr Issues Mol. Biol. 3(4):71-8.
Lindenbach B D, and Rice C M. (2002) RNAi targeting an animal virus: news from the front. Mol Cell. 9(5):925-7.
Maine, E. M. (2001) RNAi As a tool for understanding germline development in Caenorhabditis elegans: uses and cautions. Dev Biol 239; 177-89.
Ueda R. (2001). Rnai: a new technology in the post-genomic sequencing era. J Neurogenet. 15(3-4):193-204.
Ullu E, Djikeng A, Shi H, and Tschudi C. (2002) RNA interference: advances and questions. Philos Trans R Soc Lond B Biol Sci. 29; 357(1417):65-70.

1. A method of making double stranded RNA having a selected sequence comprising the steps of:
a) forming an admixture of an RNA dependent RNA polymerase, reagents for the synthesis of transcript nucleic acids, and at least one template nucleic acid, said template nucleic acid acting as a template for the synthesis of RNA encoding said selected sequence upon the imposition of nucleic acid synthesis conditions and in the presence of said reagents and RNA dependent RNA polymerase;
b) imposing nucleic acid synthesis conditions on said admixture to form an amplification product comprising double stranded RNA encoding the selected sequence.

2. The method of claim 1 wherein said template nucleic acid is a deoxyribonucleic acid.

3. The method of claim 1 wherein said RNA dependent RNA polymerase is Q-Beta replicase and modifications thereto.

4. The method of claim 1 wherein said reaction product comprising double stranded RNA inhibits the expression of a selected gene in a cell.

5. The method of claim 1 wherein said template nucleic acid has portions represented by the formula:
5′ A-B-C 3′
wherein at least one letter A and C represents a sequence recognized by said RNA dependent RNA polymerase and at least one of said A and C represents the antisense of said sequence recognized by said RNA dependant RNA polymerase, and the letter B represents a sequence corresponding to the selected sequence of the antisense of said selected sequence.

6. The method of claim 5 wherein said sequence represented by A and C are synthesized with the sequence represented B.

7. The method of claim 5 wherein said sequence represented by A and C are cloned to the sequence represented by B.

8. The method of claim 5 wherein said template is a deoxyribonucleic acid.

9. The method of claim 8 wherein said admixture further comprises a DNA-dependent RNA polymerase, said T7 DNA-dependent RNA polymerase transcribing said template to make at least one RNA recognized by said RNA dependent RNA polymerase which RNA dependent RNA polymerase generates an amplification product.

10. The method of claim 1 wherein said reagents for the synthesis of nucleic acid comprise modified nucleotides.

11. The method of claim 10 wherein said modified nucleotides have modifications at the number two position.

12. The method of claim 11 wherein said modified nucleotides comprise limited 2′-amino, 2′-fluoro, 2′-azido, 2′Omethyl, 2′ ara.

13. The method of claim 1 wherein said amplification product is used for RNAi.

14. A kit for making double stranded RNA comprising:
a) an RNA dependent RNA polymerase which RNA dependent RNA polymerase synthesizes double stranded nucleic acid in the presence of reagents;
b) reagents for the synthesis of transcript nucleic acids;
c) means for making at least one template nucleic acid, said template nucleic acid acting as a template for the synthesis of RNA encoding said selected sequence upon the imposition of nucleic acid synthesis conditions and in the presence of said reagents and RNA dependent RNA polymerase;
d) instructions for imposing nucleic acid synthesis conditions on said admixture to form an amplification product comprising double stranded RNA encoding the selected sequence.

15. The kit of claim 14 wherein said template nucleic acid is a deoxyribonucleic acid.

16. The kit of claim 14 wherein said RNA dependent RNA polymerase is Q-Beta replicase and modifications thereto.

17. The kit of claim 14 wherein said reaction product comprising double stranded RNA inhibits the expression of a selected gene in a cell.

18. The kit of claim 14 wherein said template nucleic acid has portions represented by the formula:
5′ A-B-C 3′
wherein at least one letter A and C represents a sequence recognized by said RNA dependent RNA polymerase and at least one of said A and C represents the antisense of said sequence recognised by said RNA dependant RNA polymerase, and the letter B represents a sequence corresponding to the selected sequence of the antisense of said selected sequence.

19. The method of claim 18 wherein said sequence represented by A and C are synthesized with the sequence represented B.

20. The kit of claim 18 wherein said sequence represented by A and C are cloned to the sequence represented by B.

21. The kit of claim 18 wherein said template is a deoxyribonucleic acid.

22. The kit of claim 18 wherein said admixture further comprises a DNA-dependent RNA polymerase, said DNA-dependent RNA polymerase transcribing said template to make at least one RNA recognized by said RNA dependent RNA polymerase which RNA dependent RNA polymerase generates an amplification product.

23. The kit of claim 14 wherein said reagents for the synthesis of nucleic acid comprise modified nucleotides.

24. The kit of claim 23 wherein said modified nucleotides have modifications at the number two position.

25. The kit of claim 24 wherein said modified nucleotides comprise limited 2′-amino, 2′-fluoro, 2′-azido, 2′Omethyl, 2′ ara.

26. The kit of claim 14 wherein said amplification product is used for RNAi.
